(19) United States
(12) Patent Application Publication
(10) Pub. No.: US 2015/0344968 A1
(43) Pub. Date: Dec. 3, 2015

(54) METHODS FOR DETERMINING PARP INHIBITOR AND PLATINUM RESISTANCE IN CANCER THERAPY

(71) Applicant: Institute for Cancer Research d/b/a The Research Institute of Fox Chase Cancer Center, Philadelphia, PA (US)

(72) Inventor: Neil Johnson, Philadelphia, PA (US)

(21) Appl. No.: 14/726,682

(22) Filed: Jun. 1, 2015

Related U.S. Application Data
(60) Provisional application No. 62/004,960, filed on May 30, 2014.

(51) Int. Cl. C12Q 1/68; A61K 31/55; A61K 33/24; A61K 31/704; G01N 33/574; A61K 31/502

(52) U.S. Cl. CPC C12Q 1/6886; G01N 33/57484; A61K 31/502; A61K 33/24; A61K 31/704; A61K 31/55; C12Q 2600/156; C12Q 2600/106; G01N 2333/4704

(57) ABSTRACT

Systems and methods for determining whether a cancer patient may respond to PARP inhibitor and/or platinum chemotherapy based on identifying exon excision variants in the BRCA 1 gene are provided. Exon excision variants may encode a hypomorphic BRCA1 protein. The cancer patient may be a breast cancer patient or an ovarian cancer patient. The patient may have any cancer in which exon deficiency in the BRCA1 gene contributes to resistance to PARP inhibitor or platinum therapy.

### CROSS REFERENCE TO RELATED APPLICATIONS

This application claims priority to U.S. Provisional Application No. 62/004,960, filed on May 30, 2014, the contents of which are incorporated by reference herein, in their entirety and for all purposes.

### REFERENCE TO A SEQUENCE LISTING

This application includes a Sequence Listing submitted electronically as a text file named BRCA1_CDNA_ST25.txt, created on May 22, 2014 with a size of 55,000 bytes. The Sequence Listing is incorporated by reference herein.

### FIELD OF THE INVENTION

The invention relates generally to the field of cancer diagnostics. More particularly, the invention relates to systems and methods for screening cancer patients for variants of the BRCA1 gene, especially exon-deficient variants, which induce resistance to PARP inhibitors and to platinum-containing chemotherapeutic agents.

### BACKGROUND OF THE INVENTION

Various publications, including patents, published applications, accession numbers, technical articles and scholarly articles are cited throughout the specification. Each of these cited publications is incorporated by reference, in its entirety and for all purposes, in this document.

The Breast Cancer 1 (BRCA1) protein functions in homologous recombination (HR), a vital DNA repair process that uses the undamaged sister chromatid to carry out high fidelity repair of DNA double strand breaks (DSBs). The BRCA1 gene consists of 24 exons, the largest of which is exon 11. Exon 11 frameshift mutations are commonly found in the germline of patients with hereditary breast and ovarian cancer

BRCA1 germline mutation carriers have an increased risk of developing epithelial ovarian cancer (EOC) and triple negative breast cancer (TNBC). Germline mutations in the BRCA1 and BRCA2 tumor suppressor genes are the strongest known genetic risk factors for both breast and EOC. BRCA mutations are found in 6% to 15% of women with EOC and 5% to 7% of all cases of breast cancer. Mutations are commonly insertions or deletions resulting in mRNA reading frameshifts that prematurely terminate the protein. The clinical characteristics among BRCA1 carriers differ from those of non-carriers. BRCA1-related disease is more likely to be of serous and triple-negative histology, high grade, and advanced stage.

The mutations resulting in loss of BRCA1 activity tend to sensitize cells to DNA damaging agents. Cells that are deficient in HR DNA repair, such as those lacking functional BRCA1 or BRCA2, are highly sensitive to platinum agents (cisplatin, carboplatin, or oxaloplatin) and poly(ADP-ribose) polymerase (PARP) inhibitors such as olaparib and rucaparib.

Although most BRCA1 mutant tumors initially respond well to chemotherapy, drug resistance invariably emerges and chemotherapy-resistant disease is the primary cause of death. PARP inhibitors are currently in advanced phase clinical trials and provide improved progression free survival (PFS) for BRCA mutation carriers, with the impact on overall survival (OS) under evaluation. Similar to platinum-based treatments, the efficacy of PARP inhibitor therapy is hampered by the short duration of response and acquisition of drug resistance. At present, an understanding of platinum and PARP inhibitor resistance in patient tumors is incomplete

In early phase clinical trials of PARP inhibitors, patients that harbored germline BRCA1 or BRCA2 mutations had generally improved outcomes compared to those that did not contain BRCA mutations. However, emerging data indicate that PARP inhibitor therapy may benefit only a subset of BRCA mutation carriers. Recent studies demonstrate that of the germline BRCA mutation carriers, 9 out of 17 treated with olaparib and 10 out of 26 treated with niraparib had partial responses; the remainder showed stable disease or disease progression (Gelmon, K A et al. (2011) Lancet Oncol. 12:852-61, and Sandhu, S K et al. (2013) Lancet Oncol. 14:882-92). Furthermore, similar to platinum therapies, patients who initially responded eventually developed resistance and disease progression. To improve outcomes and guide therapy, there remains a need to understand the mechanisms of resistance and identify ways around the resistance.

### SUMMARY OF THE INVENTION

The disclosure features systems for screening cancer patients for the likelihood of responding to PARP inhibitor therapy and/or platinum therapy. The systems comprise a data structure comprising structures or sequences of one or more BRCA1 genes encoding a BRCA1 protein that induces resistance to one or more PARP inhibitors or to one or more platinum-containing agents, or portion thereof, and a processor operably connected to the data structure. The processor preferably is programmed to compare a structure or sequence of a BRCA1 gene obtained from the cancer patient with the one or more BRCA1 gene structures or sequences of the data structure, and is preferably also programmed to determine a PARP inhibitor therapy response score and/or a platinum therapy response score as a result of the comparison. The system may also comprise computer readable media comprising executable code for causing the processor to compare a structure or sequence of the BRCA1 gene obtained from a cancer patient with one or more BRCA1 genes of the data structure, and for causing the processor to determine a PARP inhibitor therapy response score or a platinum therapy response score as a result of the comparison. The system may comprise an input for entering patient BRCA1 gene sequence or structure information into the system. The system may comprise an output for providing the PARP inhibitor therapy response score and/or a platinum therapy response score to a user. The system may comprise a computer network. In some preferred aspects, BRCA1 gene obtained from the patient and/or the BRCA1 genes of the data structure may comprise mRNA or a cDNA obtained from mRNA.

The disclosure also features methods for screening cancer patients for the likelihood of responding to PARP inhibitor therapy or platinum therapy. In some aspects, the methods comprise comparing a structure or sequence of a BRCA1 gene obtained from a cancer patient with one or more BRCA1 genes encoding a BRCA1 protein that induces resistance to one or more PARP inhibitors or to one or more platinum-containing agents, or portion of said protein, with a processor programmed to compare the patient-obtained sequences or structures of the BRCA1 gene with BRCA1 genes encoding a BRCA1 protein that induces resistance to one or more PARP inhibitors or to one or more platinum-containing agents, or portion thereof, and determining whether the patient will respond to PARP inhibitor therapy or platinum therapy based on the comparison. In some preferred aspects, BRCA1 gene obtained from the patient and/or the BRCA1 genes encoding a BRCA1 protein that induces resistance to one or more PARP inhibitors or to one or more platinum-containing agents may comprise mRNA or a cDNA obtained from mRNA. The methods may comprise treating the patient with a PARP inhibitor chemotherapy regimen and/or a platinum-containing agent chemotherapy regimen if the patient is determined to have a likelihood of responding positively to PARP inhibitor therapy and/or to platinum therapy e.g., because the patient does not have an exon-deficient variant of the BRCA1 gene that underlies resistance to PARP inhibitors or platinum chemotherapeutic agents. The methods may comprise avoiding treating the patient with a PARP inhibitor chemotherapy regimen and/or a platinum-containing agent chemotherapy regimen if the patient is determined not to have a likelihood of responding positively to PARP inhibitor therapy and/or to platinum therapy, e.g., because the patient has an exon-deficient variant of the BRCA1 gene that underlies resistance to PARP inhibitors or platinum chemotherapeutic agents.

In some aspects, the methods comprise entering a structure or sequence of the BRCA1 gene determined from a cancer patient into a system, for example, a system as described or exemplified herein, causing the processor of the system to compare the determined structure or sequence with the one or more BRCA1 genes encoding a BRCA1 protein that induces resistance to one or more PARP inhibitors or to one or more platinum-containing agents, or portion thereof, in the data structure of the system, and causing the processor to determine a PARP inhibitor therapy response score or a platinum therapy response score as a result of the comparison from the comparison. In some preferred aspects, BRCA1 gene obtained from the patient and/or the BRCA1 genes of the data structure may comprise mRNA or a cDNA obtained from mRNA.

The disclosure also features methods for treating breast cancer or ovarian cancer in a patient in need thereof. In some aspects, the methods comprise determining whether a BRCA1 gene obtained from the patient encodes a BRCA1 protein, or portion thereof, that induces resistance to one or more PARP inhibitors or to one or more platinum-containing agents. If the BRCA1 gene encodes a BRCA1 protein, or portion thereof, that induces resistance to one or more PARP inhibitors or to one or more platinum-containing agents, the method comprises treating the patient with a cancer treatment regimen that does not include the one or more PARP inhibitors or the one or more platinum-containing agents. If the BRCA1 gene does not encode a BRCA1 protein, or portion thereof, that induces resistance to one or more PARP inhibitors or to one or more platinum-containing agents, the method comprises treating the patient with a cancer treatment regimen that includes the one or more PARP inhibitors or the one or more platinum-containing agents.

Any of the methods may further comprise determining the sequence or structure of the BRCA1 gene obtained from the patient. Any of the methods may further comprise isolating or obtaining the BRCA1 gene from the patient.

In any of the systems or methods, the cancer patient may be any cancer patient for which PARP inhibitor therapy and/or platinum therapy may be an appropriate course of treatment. Breast cancer and ovarian cancer patients are preferred. The PARP inhibitors may comprise any combination of one or more of iniparib, olaparib, niraparib, rucparib, veliparib, BMN 673, CEP 9722, MK 4827, or E 7016. The platinum-containing agents may comprise any combination of one or more of cisplatin, carboplatin, or oxaliplatin.

The one or more BRCA1 genes may comprise an exon-deficient BRCA1 gene comprising a partial deletion of one or more exons of the BRCA1 gene. The partial deletion may comprise a partial deletion of exon 11 (exon 10b) of the BRCA1 gene. The partial deletion of exon 11 may produce a truncated nucleic acid sequence of exon 11 having SEQ ID NO: 27. The exon-deficient BRCA1 gene may comprise BRCA1-Δ11q. The BRCA1-Δ11q may comprise the nucleic acid sequence of SEQ ID NO: 26. The BRCA1 protein may comprise the amino acid sequence of SEQ ID NO: 28. The BRCA1 protein encoded by an exon-deficient BRCA1 gene may comprise a hypomorphic BRCA1 protein.

The one or more BRCA1 genes may comprise an exon-deficient BRCA1 gene comprising a complete deletion of one or more exons of the BRCA1 gene. The complete deletion may comprise a complete deletion of exon 11 (exon 10b) of the BRCA1 gene. The Complete deletion of exon 22 in the BRCA1 gene may comprise BRCA1-Δ11. The BRCA1-Δ11 may have the nucleic acid sequence of SEQ ID NO: 24. The BRCA1 protein may comprise the amino acid sequence of SEQ ID NO: 25. The BRCA1 protein encoded by an exon-deficient BRCA1 gene may comprise a hypomorphic BRCA1 protein.

### BRIEF DESCRIPTION OF THE DRAWINGS

FIG. 1 shows a summary of the analysis steps. Step 1, BRCA1 mutant tumors are assessed for BRCA1 splice variant mRNA and protein expression; Step 2, BRCA1 variant cDNA are cloned and overexpressed in BRCA1-deficient cancer cell lines; Step 3, The effect of variant overexpression on HR DNA repair and sensitivity to anti-cancer therapeutics is assessed in vitro and in vivo; Step 4, the ability of BRCA1 variants to be utilized as therapeutic biomarkers is assessed, so that patients receive personalized treatment regimens optimally designed for their tumor sub-type.

FIG. 2 shows BRCA1 exon 11 splice variants that restore the reading frame. Exon 11 mutations result in stop codons (fsX) and truncated proteins. However, removal of the mutant exon by alternative splicing restores the reading frame.

FIGS. 3A-C shows BRCA1 exon 11 mutant cell lines preferentially express the BRCA1-Δ11 isoform. FIG. 3A shows MDA-MB-231 (WT), MCF7 (WT), L56BRC1 (1806C>T), SUM149 (2288delT), UWB1.289 (2594delC), SUM1315 (185delAG) HCC1395 (5251C>T), MDA-MB-436 (5396+1G>A) cells characterized for BRCA1 isoform expression by RT-PCR analyses. The BRCA1-Δ11; -Δ9,10,11; -full length; -Δ9,10 were measured. FIG. 3B shows cell lysates were collected and BRCA1 protein detected using either N- or C-terminal specific BRCA1 antibodies. Predicted molecular weight positions for full length and Δ11 BRCA1 isoforms are denoted (*). FIG. 3C shows cells were treated with 10 Gy IR and BRCA1, RAD51, γ-H2AX foci measured by immunofluorescence.

FIGS. 4A-C show BRCA1 exon 11 mutant cell lines are less sensitive to PARP inhibitor treatment. FIG. 4A shows colony formation of cells treated with increasing concentrations of rucaparib, the LC₅₀ values (concentration required to reduce colony formation by 50%) are shown. FIG. 4B shows MCF7 (WT), MDA-MB-436 (no detectable BRCA1 protein), UWB1.289 (2594delC exon 11 expressing BRCA1 protein) were cultured in the presence of 100 nM rucaparib and cell number counted every 5 days. (Inset) Western blot of BRCA1 protein levels from UWB1.289 parent and rucaparib resistant cells (RR), cytoplasmic (c) and nuclear extract (n). FIG. 4C shows UWB1.289 rucaparib resistant cells were treated with non-target (NT) or 2 different BRCA1 targeting shRNA's, exposed to rucaparib and colony formation measured.

FIGS. 5A-D show BRCA1 2594delC provides PARP inhibitor resistance. FIG. 5A shows MDA-MB-436 cells were infected with GFP or HA-BRCA1 2594delC constructs and cultured in the presence of rucaparib. Resistant colonies were counted 1 month from treatment. FIG. 5B shows GFP, 2594delC, and 2594delC rucaparib resistant (RR) cells cytoplasmic (c) and nuclear (n) extracts, immunoblotting for HA and tubulin. FIG. 5C shows 2594delC RR cells were treated with non-target (NT) or BRCA1 shRNA, rucaparib and colony formation assessed. FIG. 5D shows GFP, HA-BRCA1 wild-type (WT) and HA-BRCA1 2594delC RR MDA-MB436 cells were subject to immunoprecipitation using anti-HA and Western blotted with indicated antibodies.

FIGS. 6A and B show BRCA1 mutant PDX models treated with PARP inhibitor. PDX124 (TNBC) and PDX196 (EOC) harbor a 2080delA BRCA1 mutation. FIG. 6A shows PDX124 tumors were initially sensitive to olaparib but several tumors developed resistance, however, PDX196 was olaparib resistant at the beginning of treatment. FIG. 6B shows Western blot analyses using N-terminal (M110), C-terminal (D9) and middle (07-434) region BRCA1 specific antibodies. 231=MDA-MB-231 BRCA1 WT cell line control, S=olaparib sensitive tumor, R=olaparib resistant tumor.

FIG. 7 shows Kaplan-Meier estimates of cumulative survival according to BRCA1 mutation group. Data were evaluated for 2,216 participants (447 BRCA1 mutation carriers and 1,769 non-carriers). Among the participants with BRCA1 mutation, 190 (42%) had mutations classified as group 1 (N-terminal fs), 128 (29%) group 2 (exon 11 fs), 48 (11%) group 4 (BRCT fs) and 81 (18%) group 5 (missense mutations from all regions).

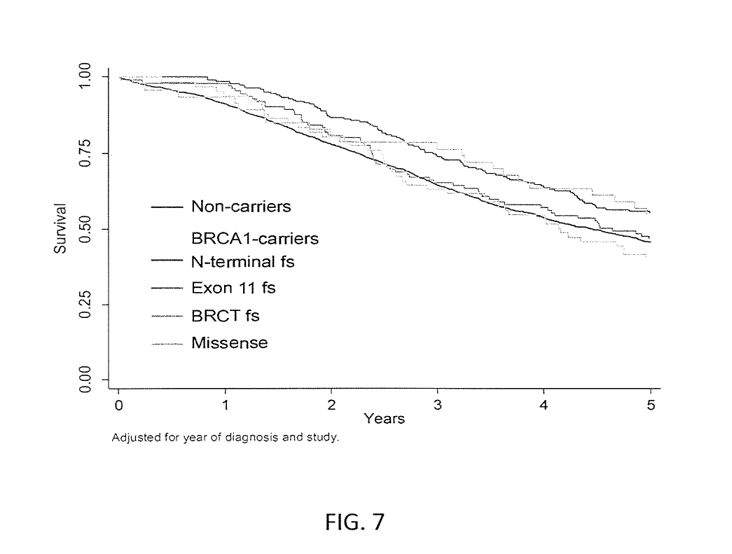

### DETAILED DESCRIPTION OF THE INVENTION

Various terms relating to aspects of the present invention are used throughout the specification and claims. Such terms are to be given their ordinary meaning in the art, unless otherwise indicated. Other specifically defined terms are to be construed in a manner consistent with the definition provided in this document.

As used throughout, the singular forms “a,” “an,” and “the” include plural referents unless expressly stated otherwise.

A molecule such as a polynucleotide or gene has been “isolated” if it has been removed from its natural environment and/or altered by the hand of a human being.

Nucleic acids include any chain of at least two nucleotides, which may be unmodified or modified RNA or DNA, hybrids of RNA and DNA, and may be single, double, or triple stranded.

A gene comprises any sequence of nucleic acids encoding a polypeptide of any length, and may be in the form of genomic DNA, mRNA, cDNA, or other non-genomic DNA form.

The terms subject and patient are used interchangeably. A subject may be any animal, and preferably is a mammal. A mammalian subject may be a farm animal (e.g., sheep, horse, cow, pig), a companion animal (e.g., cat, dog), a rodent or laboratory animal (e.g., mouse, rat, rabbit), or a non-human primate (e.g., old world monkey, new world monkey). Human beings are highly preferred.

As used herein, “exon 11” refers to what the National Center for Biotechnology Information (NCBI) designates exon 10b in Genbank Accession No. NG_{—}005905.1 and Genbank Accession No. NG_{—}005905.2. NCBI exon 10b and, thus, exon 11 has the nucleic acid sequence of SEQ ID NO: 9. Delta-11q, which is a shortened version of NCBI exon 10b, has the nucleic acid sequence of SEQ ID NO: 27.

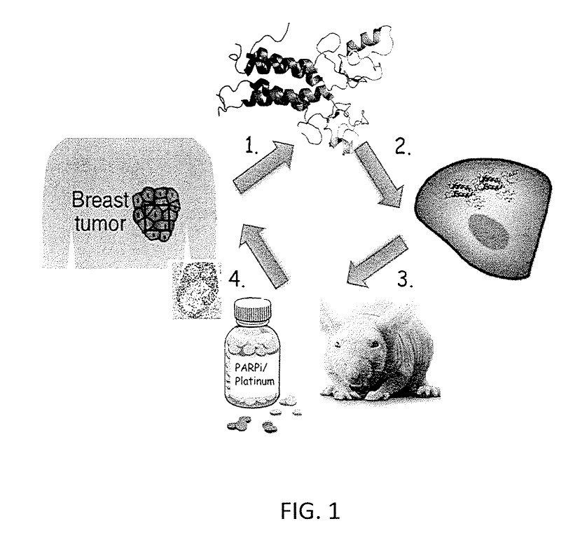

It has been observed in accordance with the invention that exon 11-mutant nascent BRCA1 mRNA molecules preferentially splice and remove the deleterious exon to restore the correct reading frame, thus generating a protein containing the N- and C-terminal, but without portions of the middle, of the full-length protein. In addition, it was observed that exon 11-deficient BRCA1 protein levels increase in response to prolonged PARP inhibitor exposure. Without intending to be limited to any particular theory of mechanism of action, it is believed that exon excision and frame correction based on alternative mRNA splicing may offer a general mechanism to produce hypomorphic, but functional BRCA1 proteins, that can provide PARP inhibitor and platinum resistance in at least breast and ovarian cancers that harbor BRCA1 mutations. It is further believed that multiple exons may be spliced out of the mature mRNA, with the resulting proteins being capable of activating homologous recombination (HR) DNA repair and inducing, supporting, and/or enhancing drug resistance. Thus, exon excision variants of BRCA1 may serve as clinical biomarkers or guideposts concerning the response to PARP inhibitor or platinum therapy (FIG. 1). Accordingly, the invention features systems and methods for determining whether a cancer patient, especially an ovarian cancer patient or breast cancer patient, will respond positively to treatment with PARP inhibitors or platinum-containing agents. Any of the methods may be carried out in vivo, in vitro, or in situ.

In some aspects, a system comprises a data structure, which comprises one or more BRCA1 genes encoding a BRCA1 protein, or portion thereof, that induces resistance to one or more PARP inhibitors or to one or more platinum-containing agents, and a processor operably connected to the data structure. The processor is programmed to compare a structure or sequence of a BRCA1 gene obtained from a cancer patient with the one or more BRCA1 genes of the data structure, and is programmed to determine a PARP inhibitor therapy response score or a platinum therapy response score as a result of the comparison. Responsiveness includes, for example, killing of cells in the tumor that come in contact with a PARP inhibitor or platinum-containing agent. The processor may comprise a computer processor. The system may comprise a computer network connection, for example, an Internet connection. The processor may comprise various inputs and outputs.

In some aspects, a system comprises a data structure, which comprises one or more BRCA1 proteins, or portion thereof, that induces resistance to one or more PARP inhibitors or to one or more platinum-containing agents, and a processor operably connected to the data structure. The processor is programmed to compare a structure of a BRCA1 protein, or portion thereof, obtained from a cancer patient with the one or more BRCA1 proteins, or portion thereof, of the data structure, and is programmed to determine a PARP inhibitor therapy response score or a platinum therapy response score as a result of the comparison. Responsiveness includes, for example, killing of cells in the tumor that come in contact with a PARP inhibitor or platinum-containing agent. The processor may comprise a computer processor. The system may comprise a computer network connection, for example, an Internet connection. The processor may comprise various inputs and outputs. The BRCA 1 protein, or portion thereof, may be hypomorphic, and may be missing amino acids that would have been encoded by an exons excised from the BRCA1 mRNA. The data structure may optionally comprise one or more BRCA1 reference proteins that represent a lack of missing amino acids (e.g., wild type or normal structure), or that are missing amino acids that have no known role in inducing resistance to one or more PARP inhibitors or to one or more platinum-containing agents.

The BRCA 1 genes of the data structure may comprise one or more exon-deficient BRCA1 genes encoding a hypomorphic BRCA1 protein, or portion thereof, that induces resistance to one or more PARP inhibitors or to one or more platinum-containing agents. Exon-deficient genes include, but are not limited to those in which one or more entire exons have been excised, as well as those in which one or more portions of one or more exons have been excised, though the remaining portions of the genes are spliced together. Preferably, the remaining portions of the genes are spliced together in frame. The BRCA1 genes of the data structure may comprise one or more portions of the full-length BRCA1 gene, for example, portions proximal to and including regions 5′ and 3′ of the missing exon, or portion thereof, where the gene, sans exon or portion thereof, has been spliced together. The data structure may optionally comprise one or more BRCA1 reference genes that represent a lack of exon deficiency in the gene (e.g., wild type or normal structure), or that encode structural alterations in BRCA1 that have no known role in inducing resistance to one or more PARP inhibitors or to one or more platinum-containing agents.

The reference genes may function, for example, as a type of control or standard against which the patient samples may be additionally or alternatively compared, via the processor, in order to determine the PARP inhibitor therapy response score or a platinum therapy response score. For example, if the comparison of patient samples with the genes in the data structure reveals that the patient does not have any of the BRCA1 exon deficiencies in the data structure, it may be determined whether the patient has a normal BRCA1 gene or an altered BRCA1 gene that nevertheless does not encode a BRCA1 protein, or portion thereof, that induces resistance to one or more PARP inhibitors or to one or more platinum-containing agents. Thus, the processor may optionally be programmed to compare patient samples with any combination of such reference genes in the data structure.

It is contemplated that the data structure includes known exon deficiencies in the BRCA1 gene, which encode a BRCA1 protein that induces resistance to one or more PARP inhibitors or to one or more platinum-containing agents, including a hypomorphic BRCA1 protein, and that the data structure may include newly identified exon deficiencies. Thus, as the knowledge in the art concerning relevant exon deficiencies advances, it is contemplated that the systems and data structures described and exemplified herein should include newly identified or characterized exon deficiencies. The data structure may include any exon deficiency described or exemplified herein.

The data structure may comprise nucleic acid sequences of BRCA1 genes, including the sequence of the full-length gene, or any portion thereof in which an exon deficiency is present, or a corresponding portion in which an exon deficiency is not present. Thus, the one or more BRCA1 genes in the data structure may comprise a nucleic acid sequence. The BRCA1 genes in the data structure may comprise genomic DNA, a non-genomic form of DNA, mRNA, or a cDNA obtained from mRNA.

Preferably, the processor is programmed to compare a sequence or structure of a BRCA1 gene, or portion thereof, determined from a cancer patient, with the BRCA1 genes, or portions thereof, in the data structure, and is also programmed to determine whether the tumor in the patient is sensitive, including the degree of sensitivity, or resistant to treatment with one or more PARP inhibitors and/or with one or more platinum-containing agents. For example, the processor may be programmed to determine a PARP inhibitor therapy response score and/or a platinum therapy response score as a result of the comparison of the BRCA1 gene sequence or structure in the patient with the BRCA1 gene sequences or structures in the data structure. Thus, for example, once the sequence or structure of the BRCA1 gene in the patient is determined, the sequence or structure may be entered into the system, and the patient's BRCA1 gene sequence or structure may then be compared against the BRCA1 gene sequences and/or structures in the data structure, and if the patient is determined to have, or not have, an exon deficiency in the BRCA1 gene, a likelihood of responsiveness of the patient's tumor to treatment with either or both of PARP inhibitors or platinum-containing agents can be determined. The BRCA1 gene from the patient may comprise genomic DNA, a non-genomic form of DNA, mRNA, or a cDNA obtained from mRNA.

The processor may determine a PARP inhibitor therapy response score and/or a platinum therapy response score based on the comparison of the patient-sample BRCA1 gene with the BRCA1 genes in the data structure. The determined response score may then be provided to a user, for example, a medical practitioner or the cancer patient. Accordingly, in some aspects, the system optionally comprises an output for providing the PARP inhibitor therapy response score and/or a platinum therapy response score to a user.

The form of the PARP inhibitor therapy response score and/or a platinum therapy response score is not critical, and may vary according to the needs of the practitioner, patient, or user of the system. In its simplest form, such a response score may be an indication whether the cancer patient, whose samples have been entered into the system for comparison against the data structure, will or will not respond positively to chemotherapy with PARP inhibitors and/or with platinum-containing agents. A positive response includes, for example, a clinically significant killing of tumor cells, including a reduction in the size of the solid tumor, and including elimination of the tumor. A positive response may also include, for example, stabilizing the cancer such that no further growth occurs. At least a partial positive response may be considered a beneficial treatment outcome. A response score may comprise a scale of a likely positive response, for example, a scale of 1 to 10 or other suitable integers, with one end of the spectrum corresponding to a score that the patient likely will not respond positively to PARP inhibitor and/or platinum-based chemotherapy, and the other end of the spectrum corresponding to a score that the patient likely will respond positively to PARP inhibitor and/or platinum-based chemotherapy. A response score may comprise a value indicative of a high likelihood of a positive response to PARP inhibitor and/or platinum-based chemotherapy, a value indicative of a moderate likelihood of a positive response to PARP inhibitor and/or platinum-based chemotherapy, or a value indicative of a low likelihood of a positive response to PARP inhibitor and/or platinum-based chemotherapy. In some aspects, a response score may be backed up by statistical significance, according to any suitable statistical methodology.

A response score may, for example, be a function of the number and/or location of the exon deficiency or deficiencies in the patient's BRCA1 gene, as well as whether or not an exon deficiency results in the expression of a hypomorphic BRCA1 protein, or in the expression of a BRCA1 protein that induces resistance to one or more PARP inhibitors or to one or more platinum-containing agents. A response score may, for example, be a function of the type of chemotherapy, including the particular type of PARP inhibitor, or the particular type of platinum-containing agent, or whether multiple PARP inhibitors are used in combination, or whether multiple platinum-containing agents are used in combination, or whether PARP inhibitors and platinum-containing agents are used in combination.

The PARP inhibitors may comprise iniparib, olaparib, niraparib, rucparib, veliparib, BMN 673, CEP 9722, MK 4827, E 7016, or any combination thereof. The platinum-containing agents may comprise cisplatin, carboplatin, oxaliplatin, or any combination thereof.

In some aspects, the processor may be programmed to recommend a particular treatment regimen for the patient, based on the PARP inhibitor therapy response score and/or platinum therapy response score. For example, the processor may recommend for patients who are determined to have a strong likelihood of a positive response that such patients be administered one or more PARP inhibitors and/or one or more platinum-containing agents. The processor may also recommend, for example, for patients who are determined to have a low likelihood of a positive response that such patients not be administered PARP inhibitors or platinum-containing agents in favor of an alternative chemotherapeutic regimen. In this way, time otherwise spent on an ineffective therapy can be devoted to more promising therapies, and unnecessary untoward effects can be avoided. The chemotherapeutic regimen may be directed by a medical practitioner according to patient care standards known or suitable in the art.

Optionally, the system may comprise an input for entering into the system BRCA1 gene sequences or structures determined or otherwise obtained from patient samples. Optionally, the system may comprise an output for providing results of a comparison, including a PARP inhibitor therapy response score and/or a platinum therapy response score, to a user such as the patient, or a technician, or a medical practitioner. The BRCA1 gene may be obtained from any suitable source in the patient, including blood or buccal tissue, or tumor tissue. It is preferred that the BRCA1 gene be obtained from the tumor in the patient.

The patient may be any cancer patient in which the underlying tumors may be treated with a PARP inhibitor or a platinum-containing agent. The patient may be any cancer patient in which BRCA1 plays a role in PARP inhibitor and/or platinum agent resistance. Breast cancer patients and ovarian cancer patients are highly preferred. Epithelial ovarian cancer patients are a non-limiting example of an ovarian cancer patient. Patients are preferably human beings.

In some aspects, the system may comprise computer readable media comprising executable code for causing a programmable processor to compare the sequence or structure of the BRCA1 gene obtained from a cancer patient with BRCA1 genes in a data structure, and for causing a programmable processor to determine a PARP inhibitor therapy response score and/or a platinum therapy response score as a result of the comparison. The PARP inhibitor therapy response score and/or a platinum therapy response score may be as described above, including a likelihood that the cancer patient will or will not respond positively to PARP inhibitor and/or platinum-based chemotherapy. Such computer readable media are also featured in accordance with the invention separate from the systems of the invention. The computer readable media may comprise a processor, which may be a computer processor.

In one aspect, the invention provides methods for determining whether a cancer patient may respond positively to PARP inhibitor therapy and/or platinum therapy. In some aspects, the methods generally comprise comparing a structure or sequence of a BRCA1 gene isolated from a cancer patient with one or more BRCA1 gene structures or sequences that encode a BRCA1 protein that induces resistance to one or more PARP inhibitors or to one or more platinum-containing agents, for example, one or more sequences or structures of exon-deficient BRCA1 genes encoding a BRCA1 protein, or portion thereof, that induces resistance to one or more PARP inhibitors or to one or more platinum-containing agents, including a hypomorphic BRCA1 protein, and determining whether the patient will respond to PARP inhibitor therapy or platinum therapy based on the comparison. The methods may optionally comprise determining a structure or sequence of the BRCA1 gene from the patient. The methods may also comprise obtaining the BRCA1 gene from the patient. The patient may be breast cancer patient. The patient may be an ovarian cancer patient.

The comparing step may be carried out, for example, using a processor programmed to compare patient BRCA1 gene sequences or structures with one or more BRCA1 gene structures or sequences that encode a BRCA1 protein that induces resistance to one or more PARP inhibitors or to one or more platinum-containing agents. The one or more BRCA1 gene structures or sequences that encode a BRCA1 protein that induces resistance to one or more PARP inhibitors or to one or more platinum-containing agents may, for example, be present in a data structure. The determining step may be carried out, for example, using a processor programmed to determine whether a cancer patient will respond to PARP inhibitor therapy and/or respond to platinum therapy, based on the comparison of the patient BRCA1 gene with one or more BRCA1 gene structures or sequences that encode a BRCA1 protein that induces resistance to one or more PARP inhibitors or to one or more platinum-containing agents. In some aspects, determining whether the patient will respond to PARP inhibitor therapy or platinum therapy comprises generating a PARP inhibitor therapy response score and/or a platinum therapy response score as a result of the comparison.

The systems, computer readable media, and platforms described or exemplified herein may be used in accordance with such methods. For example, the methods may comprise determining a structure or the sequence of the BRCA1 gene from a sample, such as a tumor cell or blood, isolated from a cancer patient, entering the determined BRCA1 sequence or structure into a system as described or exemplified herein, causing the processor of the system to compare the determined structure or sequence from the patient with BRCA1 gene structures or sequences that encode a BRCA1 protein that induces resistance to one or more PARP inhibitors or to one or more platinum-containing agents that are in the data structure, and causing the processor to determine a PARP inhibitor therapy response score and/or a platinum therapy response score based on the comparison of patient and database BRCA1 gene structures and/or sequences.

In some aspects, in which the cancer patient is determined to have a likelihood of responding positively to PARP inhibitor or platinum-based chemotherapy, the methods may further comprise the steps of treating the patient with one or more PARP inhibitors and/or one or more platinum-containing agents. Treating may include administering to the patient the agent in an amount effective to treat the tumor. Administration may be according to any suitable route.

Thus, the invention also features methods for treating a tumor in a patient in need thereof. In some aspects, the methods comprise determining whether a Breast Cancer 1 (BRCA1) gene obtained from the patient encodes a BRCA1 protein, or portion thereof, that induces resistance to one or more PARP inhibitors or to one or more platinum-containing agents. In some aspects, the methods comprise comparing a structure or sequence of a BRCA1 gene obtained from the patient with one or more BRCA1 gene structures or sequences that encode a BRCA1 protein that induces resistance to one or more PARP inhibitors or to one or more platinum-containing agents, or portion thereof, for example, one or more sequences or structures of exon-deficient BRCA1 genes encoding a BRCA1 protein that induces resistance to one or more PARP inhibitors or to one or more platinum-containing agents, or portion thereof, including a hypomorphic BRCA1 protein. The methods may comprise isolating the BRCA1 gene from the patient and determining the sequence or structure of the gene, and based on the sequence or structure, make the determination of whether the gene encodes a BRCA1 protein, or portion thereof, that induces resistance to one or more PARP inhibitors or to one or more platinum-containing agents. The BRCA1 gene may comprises DNA or mRNA, or may comprise a cDNA obtained from mRNA such that the method may comprise converting mRNA into a cDNA.

If the BRCA1 gene encodes a BRCA1 protein, or portion thereof, that induces resistance to one or more PARP inhibitors or to one or more platinum-containing agents, the method comprises treating the patient with a cancer treatment regimen that does not include the one or more PARP inhibitors or the one or more platinum-containing agents. If the BRCA1 gene does not encode a BRCA1 protein, or portion thereof, that induces resistance to one or more PARP inhibitors or to one or more platinum-containing agents, the method comprises treating the patient with a cancer treatment regimen that includes the one or more PARP inhibitors or the one or more platinum-containing agents.

The methods primarily direct the practitioner as to whether the patient will or will not respond to PARP inhibitor- and/or platinum agent-based chemotherapeutic regimens, such that if the patient will respond, such agents may be indicated and administered or if the patient will not respond, such agents are contra-indicated such that alternative therapies may be pursued. Alternative therapies are desired in such an instance such that the patient does not waste crucial time on a chemotherapeutic regimen that is unlikely to provide much benefit and, moreover, such that the patient avoids enduring side effects from PARP inhibitors and platinum agents when such agents are unlikely to provide the patient with a therapeutic benefit.

The treatment regimen may further include surgery, radiation therapy, hormone therapy, targeted therapy, diet or nutrition modifications, and administration of other chemotherapeutic agents (other than PARP inhibitors and platinum-containing agents).

For such treatment methods, the determining step may comprise determining whether the BRCA1 gene obtained from the patient has at least a partial deletion of one or more exons of the BRCA1 gene, or may comprise determining whether the BRCA1 gene obtained from the patient has a complete deletion of one or more exons of the BRCA1 gene. A partial or complete deletion may be a partial or complete deletion of exon 11 (a.k.a., exon 10b) of the BRCA1 gene. A partial deletion of exon 11 may comprise a truncated nucleic acid sequence of exon 11 having SEQ ID NO: 27. A partial deletion of exon 11 may comprise BRCA1-Δ11q. The BRCA1-Δ11q may comprise the nucleic acid sequence of SEQ ID NO: 26. A complete deletion of exon 11 may comprise BRCA1-Δ11. The BRCA1-Δ11 may comprise the nucleic acid sequence of SEQ ID NO: 24. The BRCA1 protein may comprise the amino acid sequence of SEQ ID NO: 28 or SEQ ID NO:

The one or more PARP inhibitors may comprise any one or any combination of iniparib, olaparib, niraparib, rucparib, veliparib, BMN 673, CEP 9722, MK 4827, and E 7016. The one or more platinum-containing agents may comprise any one or any combination of cisplatin, carboplatin, and oxaliplatin.

The following examples are provided to describe the invention in greater detail. They are intended to illustrate, not to limit, the invention.

### Example 1

### Preliminary Studies

Both human and mouse cells express an alternatively spliced in-frame variant referred to as BRCA1-Δ11, lacking most or all of exon 11. Mice bearing mammary-specific deletions of exon 11 develop mammary adenocarcinomas with chromosomal instability and are sensitive to PARP inhibitor treatment. However, murine embryos bearing targeted mutations that selectively abolish expression of full-length Brca1, while leaving Brca1-Δ11 expression intact, survive significantly longer than mice expressing targeted mutations that abolish expression of both Brca1 and Brca1-Δ11. Additionally, cells expressing Brca1-Δ11 are able to form residual Brca1 and Rad51 foci. Without intending to be limited to any particular theory or mechanism of action, it is believed that Brca1-Δ11 is able to partially compensate for loss of full-length Brca1. To date, the role of BRCA1 isoforms in the development of platinum or PARP inhibitor resistance has not been addressed. These experiments investigate the impact of deleterious BRCA1 exon 11 mutations on protein function and drug resistance.

The human cancer cell lines SUM149, L56BRC1 and UWB1.289 contain exon 11 frameshift mutant BRCA1 alleles. In contrast, SUM1315 cells have an N-terminal frameshift mutation, and MDA-MB-436 and HCC1395 cells harbor C-terminal mutations resulting in BRCT domain truncations. The wild-type BRCA1 allele could not be detected in any of the 6 cell lines; however, the mutant alleles were readily detectable by Sanger sequencing.

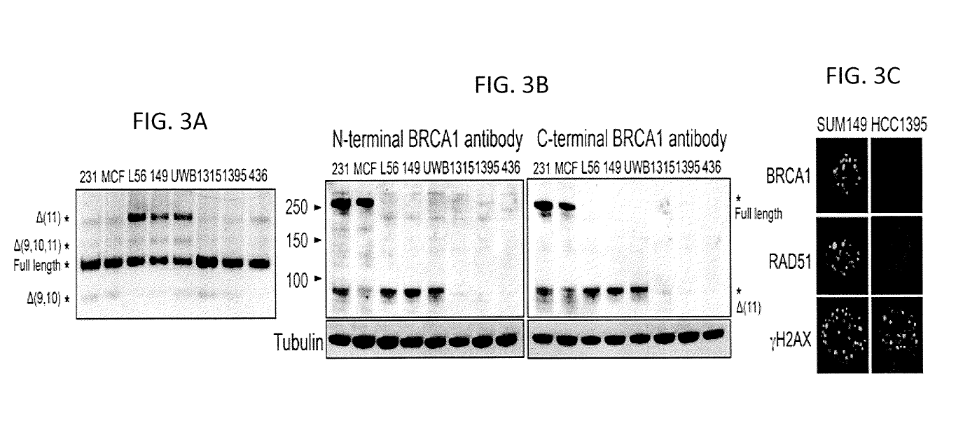

Reverse transcriptase-PCR analyses detected full length BRCA1 mRNA in BRCA1 wild-type MDA-MB-231 and MCF7 cells as well as all of the BRCA1 mutant cell lines. However, the BRCA1-Δ11 isoform mRNA could only be strongly detected in L56BRC1, SUM149 and UWB1.289 cell lines that harbor BRCA1 exon 11 frameshift mutations (FIG. 3A). Furthermore, BRCA1 wild-type cells strongly expressed full-length BRCA1 protein, but weakly expressed BRCA1-Δ11 isoform. In contrast, exon 11 mutant-containing cell lines L56BRC1, SUM149, UWB1.289 did not have detectable full-length BRCA1 protein expression, but the BRCA1-Δ11 isoform was abundant and detectable with both N- and C-terminally directed BRCA1 antibodies by Western blot, indicating the protein was not a truncated N-terminal fragment.

In contrast, no full length or BRCA1-Δ11 isoform protein could be detected in SUM1315 cells that harbor an extreme N-terminal truncating mutation, or in MDA-MB-436 and HCC1395 cells containing BRCT domain mutations, each known to result in protein destabilization and degradation (FIG. 3B). Potentially predictive of functionality, BRCA1 and RAD51 foci were readily detectable by immunofluorescence in SUM149, L56BRC1 and UWB1.289 cell lines but completely absent in SUM1315, MDA-MB-436 and HCC1395 cells (FIG. 3C).

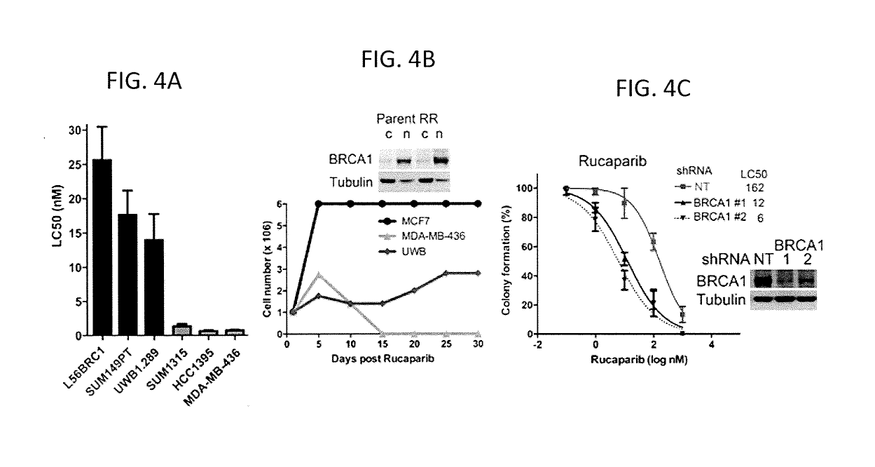

The impact of the PARP inhibitor rucaparib was assessed on the colony formation capacity of BRCA1 mutant cell lines. Cell lines that contained an exon 11 BRCA1 mutation and expressed the BRCA1-Δ11 protein were significantly more resistant to rucaparib (FIG. 4A) compared to cell lines where mutant BRCA1 protein could not be detected by Western blot. In cell growth experiments, MCF7 cells (WT BRCA1) could grow unhindered in the presence of PARP inhibitor. In contrast, MDA-MB-436 cells (undetectable mutant BRCA1 protein) quickly died. However, exon 11 deficient BRCA1 protein-expressing UWB1.289 cells quickly adapted and could grow in the presence of rucaparib. Notably, resistant cells expressed higher levels of BRCA1 protein (FIG. 4B). When BRCA1-Δ11 protein was depleted from UWB1.289 rucaparib resistant cells with shRNA, cells were re-sensitized to PARP inhibitor treatment (FIG. 4C), suggesting that the BRCA1-Δ11 protein is essential for resistance.

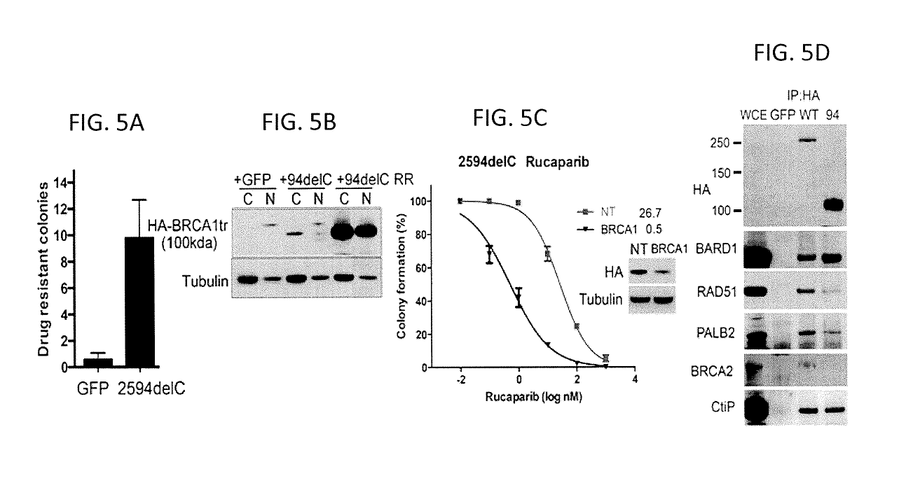

To further assess the ability of the 2594delC exon 11 mutant BRCA1 protein expressed in UWB1.289 cells to provide PARP inhibitor resistance, PARP inhibitor sensitive MDA-MB-436 cells were infected with lentivirus containing the HA-BRCA1 2594delC construct. MDA-MB-436+HA-BRCA1 2594delC cells remained exquisitely sensitive to rucaparib treatment (data not shown); however, following culture in the presence of low rucaprib concentrations to select for drug resistant colonies, cells expressing the 2594delC BRCA1 protein produced substantially more drug resistant colonies compared to cells expressing GFP alone (FIG. 5A). Resistant colonies could grow in the presence of 1 μM rucaparib and had dramatically increased expression of the 2594delC BRCA1 protein (FIG. 5B).

When the mutant BRCA1 protein was depleted using shRNA, resistant cells were re-sensitized to PARP inhibitor treatment (FIG. 5C). Furthermore, the mutant BRCA1 protein expressed in MDA-MB-436+HA-BRCA1 2594delC rucaparib resistant cells was a BRCA1-Δ11-like protein. The cloned cDNA contained the N- and C-termini, but lacked exons 6-11 of full length BRCA1. Furthermore, HA tagged 2594delC protein from MDA-MB-436+HA-BRCA1 2594delC rucaparib resistant cells was immunoprecipitated, and it strongly co-immunoprecipitated with BARD1 and CtlP, which bind the N- and C-terminal of BRCA1, respectively, but at best weakly interacted with proteins that bind to the middle of the full length BRCA1 protein, including RAD51, PALB2 and BRCA2 (FIG. 5D). These data indicate that cells containing the 2594delC BRCA1 mutant construct selected for expression of a BRCA1-Δ11-like protein that was capable of promoting drug resistance in the presence of PARP inhibitor selection pressure.

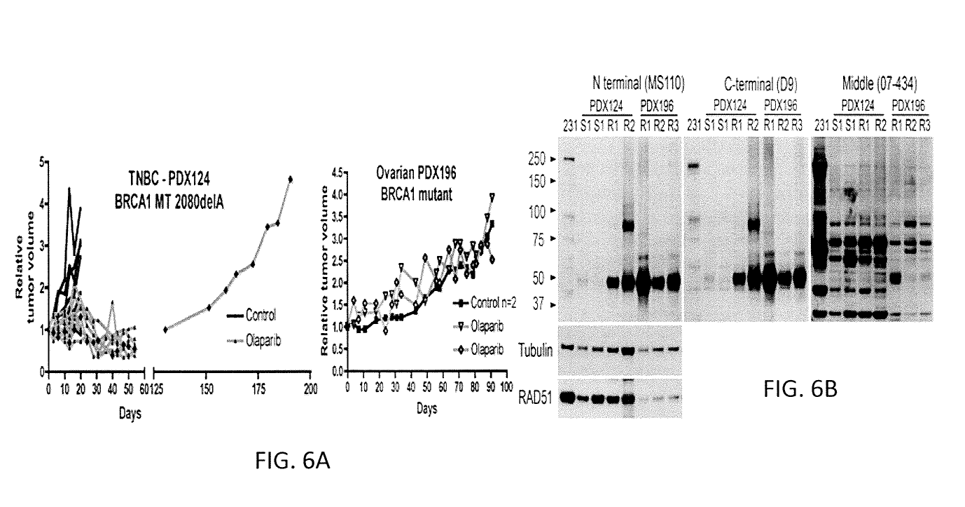

Current experiments are evaluating PARP inhibitor resistance mechanisms in PDX models of BRCA1 mutant cancer. More than 10 BRCA1 breast and ovarian PDX models are currently being derived. Initially, PDX models derived from 2 individual patients that harbored identical germline BRCA1 2080delA exon 11 mutations were evaluated. One patient had a clinical response to olaparib (PDX124), while the other patient progressed (PDX196). These clinical outcomes were reproduced in mouse models where PDX124 and PDX196 tumors were PARP inhibitor-sensitive and -resistant, respectively. However, prolonged treatment of mice harboring PDX124 resulted in the derivation of resistant tumors (FIG. 6A).

In preliminary studies, BRCA1 protein levels were measured in tumors by Western blot. BRCA1 antibodies specifically recognizing the N- and C-terminal regions of BRCA1 produced identical blots from separate membranes. However, an antibody recognizing the middle of the full length BRCA1 protein produced a range of likely non-specific bands, suggesting that proteins detected with N- and C-terminal antibodies are BRCA1 in-frame isoforms devoid of the middle region of full-length BRCA1. No BRCA1 isoforms could be detected in olaparib-sensitive PDX124 tumors. In contrast, strong bands were detected in the 50 kDa region from PDX124 and PDX196 olaparib-resistant tumors with N- and C-terminal specific BRCA1 antibodies. Because exon 11-deleted proteins are estimated to have a molecular weight of 95 kDa, it is believed that the 50 kDa band detected in resistant tumors is likely to be a product of additional exon splicing (FIG. 6B).

### Example 2

### Identification of BRCA1 Isoforms that are Highly Expressed in Drug Resistant Tumors

It is believed that in-frame splicing has the potential to remove deleterious exons from the mature mRNA to extend the reading frame through to the C-terminus. Studies in PDX BRCA1 mutant models suggest that multiple exons may be removed in drug resistant tumors (FIG. 6B), and that tumors may produce a more diverse range of splice variants in comparison to cell lines. Furthermore, it is not known if splicing of deleterious exons occurs only in exon 11 BRCA1 mutant tumors or if this mechanism of resistance is more general and applicable to other mutation types.

Experimental Methods. The mRNA and peptide sequence, as well as the expression levels of BRCA1 splice variants in mutant PDX and primary patient tumors, will be assessed and confirmed under IRB approved protocols. BRCA1 mutant tumors will be obtained from three sources so that sufficient numbers can be analyzed for variant expression. (1) The Fox Chase Cancer Center Bio-repository Facility stores well-annotated fresh frozen and paraffin-embedded BRCA1 mutant patient tumors. Twelve frozen breast and ovarian tumors from individuals with germline frameshift BRCA1 mutations have been identified. An additional 7 paraffin-embedded tumors are available if IHC analyses is warranted. There are 100+ ovarian and breast BRCA1 wild-type tumors available to serve as controls. (2) University of Washington, Seattle will supply RNA and protein from approximately 136 BRCA1 mutant tumors. Of these tumors, around half contain exon 11 mutations and 30 are paired platinum-sensitive and -resistant BRCA1 mutant patient tumors. All tumors have been extensively analyzed for pathological and biological characteristics, including reversion mutation status and clinical outcome. Currently 10 tumors from PARP inhibitor treated patients with BRCA1 mutations are available for evaluation. (3) The Vail D'Hebron Institute of Oncology, Spain, will also supply BRCA1 protein variants in BRCA1 mutant PDX and primary patient tumors. Ten BRCA1 mutant PDX and primary tumors are available for analyses. All PDX tumors have been analyzed for olaparib sensitivity.

Total RNA will be prepared from cultured cells and homogenized tumors using RNAeasy® kit (Qiagen GmbH Corp.) and cDNA generated using the SuperScript® III Reverse Transcriptase system (Life Technologies Corp.) according to manufacturer's instructions. Regions contained within exons 1 to 24 will be amplified using primers designed to capture all potential variants. Splice variants amplified will be gel purified (QIAquick® Gel Extraction Kit, Qiagen GmbH Corp) and DNA sequenced using an ABI 3130xl capillary genetic analyzer according to manufacturer's instructions.

Standard Western blot analyses of BRCA1 variants using N- and C-terminal specific antibodies (FIG. 6B) will confirm the peptide sequence of BRCA1 protein variants. Following immunopreciptation of BRCA1 isoforms, proteins will be trypsin-digested, purified using reverse-phase C18 spin columns and analyzed using LC/MS on a Q Exactive® mass spectrometer (Thermo Finnigan, LLC) equipped with the EASY-nLC 1000 system. Peptide sequences will be identified using Proteome Discoverer 1.3 (Thermo Fisher Scientific) accessing MASCOT 2.2.4 and SEQUEST databases.

Cloned isoforms will be ligated into the pENTR1A-HA-BRCA1 construct and shuttled into the pLX304 lentivirus destination vector using the LR Clonase® system (Life Technologies Corp.) and lentivirus generated using standard protocols. As in Example 1 (FIG. 5), MDA-MB-436 cells will be infected with lentivirus-containing HA-BRCA1 isoforms or GFP only constructs, and stable cell populations obtained with blastcitidine selection. MDA-MB-436 cells will be utilized for add-back experiments as these cells are exquisitely PARP inhibitor sensitive and BRCA1 protein isoform expression cannot be detected using N- or C-terminal specific antibodies. Western blot analyses will be used to assess protein levels of HA-BRCA1, using anti-HA and BRCA1 N- and C-terminal antibodies. For all experiments, MDA-MB-436 cells expressing GFP or wild-type HA-BRCA1 will be used as negative and positive controls. First, stable cell lines will be assessed for growth and viability alterations by counting cell numbers every 3 days, as well as colony formation. The effect of variant expression on colony formation capacity will be assessed after fixation and staining 2-weeks post-seeding.

If the over-expression of BRCA1 isoforms in MDA-MB-436 cells impacts cell growth or viability, the effect of protein over-expression in additional cell lines will be assessed. To rule out cell line specific variant effects, HCC1395 and SUM1315 BRCA1 mutant cell lines will be used, where BRCA1 protein expression could not be detected and cells were extremely sensitive to PARP inhibitor treatment. If variants also negatively impact growth and viability of these cell lines, the variant will be designated as unlikely to play a role in drug resistance. However, if overexpression of the variant has little impact on cell viability, the effect of variant expression on HR DNA repair and drug resistance will be assessed.

### Example 3

### Characterization of the Ability of BRCA1 Isoforms to Provide HR DNA Repair and Drug Resistance

The experiments from Example 2 will establish cell lines over-expressing novel BRCA1 variants. The following experiments will determine if variants contribute to HR DNA repair and therapy resistance.

Experimental Methods. MDA-MB-436 cells were found not to form detectable BRCA1 or RAD51 foci under any experimental conditions tested. However, the addition of wild-type BRCA1 add-back restores BRCA1 and RAD51 focus formation.

First, the ability of variants to restore BRCA1 and RAD51 focus formation will be evaluated, and compared to empty vector and wild-type BRCA1 add-back cell lines. Cells will be treated with 10 Gy γ-irradiation (IR) or 1 μM rucaparib and BRCA1 and RAD51 foci formation measured at multiple time points post-treatment by immunofluorescence. Cells will be fixed and stained with respective antibodies followed by fluorescent conjugated secondary antibodies and DAPI staining. A minimum of 300 cells will be counted for foci positive cells per treatment condition.

To directly measure the impact of variants on HR DNA repair, the U2OS-DR-GFP reporter system will be used. U2OS-DR-GFP cells will be infected with lentivirus to over-express HA-BRCA1 wild-type and variant proteins. Subsequently, the endogenous wild-type BRCA1 protein will be deleted using 3′UTR targeting BRCA1 siRNA, and the ability of exogenously expressed variants, that do not contain a 3′UTR and so are siRNA-resistant, to rescue HR defects will be assessed. Cells will be transfected with an Sce-1-expressing plasmid, and GFP levels determined 3 days post-transfection by flow cytometry.

To assess the ability of variants to provide anti-cancer therapy resistance, MDA-MB-436 cells expressing variant BRCA1 proteins will be used. Cells will be treated with increasing concentrations of the PARP inhibitors rucaparib or olaparib, and relevant cytotoxic agents cisplatin, adriamycin and taxol and colony formation assessed. LC₅₀ (Lethal concentration 50-concentration required to kill 50% of cells) concentrations will be calculated using GraphPad Prism® Software.

Phenotypes that are observed with BRCA1 variants in colony formation assays will be confirmed in vivo. MDA-MB-436 cells expressing wild-type or variant tumors will be subcutaneously implanted into athymic nu/nu mice. GFP-expressing control cells will be implanted in one flank, and variant or wild-type BRCA1 expressing cells in the other flank of the mouse to directly compare the impact of therapy on tumor growth. A total of 8 nude mice per cell line per chemotherapy will be injected. Xenograft measurements will begin at day 3 post-implantation, and will be ongoing every third day throughout duration of the experiment. After xenografts reach approximately 200 mm³ in size, 8 mice will be treated with vehicle, 8 mice with chemotherapies, to be determined based on colony assay results, and tumor growth measured.

Isoform expression on cell growth and viability will be evaluated using generalized linear models assuming appropriate family and link functions. Models will be estimated using Generalized Estimating Equations (GEE). With 6 case and 6 control cell lines, there is 80% power to detect standardized differences between groups of 1.1 assuming a simple regression, normal family, and identity link. A standardized effect is the effect after transforming variables to have standard deviation equal to one. This assumes 3 repeated measurements, a within cell line correlation of 0.2, and a 5% Type I error rate (2-sided). The detectable effect is consistent with the magnitude of differences shown in FIG. 5C. For animal studies with 8 mice per group, standardized differences of 0.96 can be detected using the same assumptions as above (e.g., 80% power, 5% Type 1 error, 3 measurements, 0.2 correlation).

If BRCA1 isoforms are incapable of providing drug resistance, secondary reversion mutations may restore wild-type BRCA1 proteins. The BRCA1 gene will be sequenced to determine if reversion mutations have occurred. Additional cooperating mutations may promote resistance. Additional studies will compare differences in gene expression and mutational status between drug sensitive and resistant cell lines and tumors. To further differentiate between endogenous and exogenous BRCA1 proteins in cDNA add-back experiments, endogenous BRCA1 functionality may be assessed using shRNA that targets the BRCA1 3′UTR to distinguish between endogenous and exogenous BRCA1 proteins, or generate silent mutations in BRCA1 cDNA constructs that provide resistance to RNAi.

### Example 4

### Summary

Clinically, platinum resistant EOC and triple negative breast cancer is a critical hurdle for patient survival. Despite platinum being routinely administered for the treatment of EOC for several decades, an understanding of drug resistance is incomplete. PARP inhibitors represent an exciting new treatment strategy for BRCA mutated cancer; however, drug resistance limits efficacy. Our work provides new insights into the current understanding of drug resistance in BRCA1 mutant disease, and will ultimately promote the development of strategies for the optimal application of platinum and PARP inhibitor therapy.

The experiments from Example 1 reveal a novel mechanism of resistance to therapy in BRCA1-mutant cell lines. The data suggest that BRCA1 exon 11 frame-shifting mutations preferentially express in-frame exon 11-deleted BRCA1 isoforms (BRCA1-Δ11), in which the C-terminal end of the protein is restored to the correct reading frame. It was observed that BRCA1 mutant cell lines harboring exon 11 mutations increase BRCA1-Δ11 protein expression in the presence of PARP inhibitor selection pressure. Furthermore, BRCA1-Δ11 proteins are believed to be important for cellular PARP inhibitor and platinum resistance. It is believed that the presence of BRCA1-Δ11 isoforms in tumors may be predictive of a more limited PARP inhibitor and platinum response in cancer patients that harbor BRCA1 mutant tumors.

The expression of BRCA1 truncated isoforms arising from alternative splicing in cell lines and tumors has been well documented, but little is known about the function or purpose of these isoform variants. Because isoforms lack essential regions of the full-length protein, it has been assumed that these proteins have a limited role in mammalian cell biology. It is believed that the successful completion of the experiments from Examples 2 and 3 above will change this paradigm.

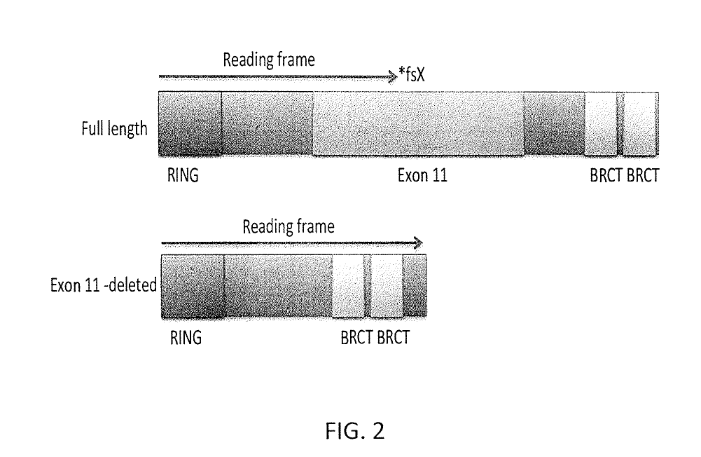

Important roles for BRCA1 protein isoforms in DNA repair and drug resistance have been uncovered, and established and novel isoforms expression and function will be characterized. BRCA1 isoforms expressed in drug resistant cells are also found in normal cells and tissues; however, BRCA1 mutant cells selectively overexpress protein isoforms that lack the exon where the deleterious mutation is located (FIG. 2). BRCA1 gene knockout is lethal. This specification provides the first direct evidence that BRCA1 truncated isoforms are in fact hypomorphic proteins and do contribute to DNA repair. Isoforms have a lower DNA repair capacity compared to the full-length BRCA1, but it is believed that highly expressed isoforms are capable of providing a threshold level of DNA repair that induces resistance to therapies that exploit HR defects.

### Example 5

### Role of 53BP1

When a double stranded break (DSB) is initially formed, BRCA1-CtlP complexes remove tumor suppressor p53-binding protein 1 (53BP1) from the DSB and activate nucleases required for DNA end resection. Later, when a DSB has been resected and single stranded DNA (ssDNA) regions have been generated, RAD51 binds to ssDNA. RAD51 foci formation is an essential event for HR DNA repair and BRCA1 is important for the direct loading of RAD51 onto ssDNA, possibly through its interaction with PALB2-BRCA2-RAD5150, 51.

Using a human breast cancer cell line that contains a BRCT domain truncating BRCA1 mutation, it was observed that stabilization of the mutant BRCA1 protein is critical for the restoration of RAD51 focus formation and PARP inhibitor and platinum resistance. The presence of stabilized BRCT domain mutant BRCA1 protein was confirmed in 2 out of 4 EOC patient tumors that had developed platinum resistance. Other mechanisms of resistance have been described in in Brca1-mutated mouse mammary tumors. Activation of P-glycoprotein, which pumps drugs out of cells, or loss of 53BP1 expression, resulting in the activation of DNA end resection and HR, provided PARP inhibitor resistance. However, 53BP1 protein expression was not found to correlate with platinum sensitivity in human patient tumors or cell lines, and resistance-causing mutations have yet to be found in patient tumors. To date, the described mechanisms of PARP inhibitor and platinum resistance occur in only a fraction of the BRCA1 mutant patient population or in Brca1-mutated mouse mammary tumors.

The invention is not limited to the embodiments described and exemplified above, but is capable of variation and modification within the scope of the appended claims.

We claim:

1. A method for treating breast cancer or ovarian cancer in a patient in need thereof, comprising determining whether a Breast Cancer 1 (BRCA1) gene obtained from the patient encodes a BRCA1 protein, or portion thereof, that induces resistance to one or more PARP inhibitors or to one or more platinum-containing agents; if the BRCA1 gene encodes a BRCA1 protein, or portion thereof, that induces resistance to one or more PARP inhibitors or to one or more platinum-containing agents, treating the patient with a cancer treatment regimen that does not include the one or more PARP inhibitors or the one or more platinum-containing agents, and if the BRCA1 gene does not encode a BRCA1 protein, or portion thereof, that induces resistance to one or more PARP inhibitors or to one or more platinum-containing agents, treating the patient with a cancer treatment regimen that includes the one or more PARP inhibitors or the one or more platinum-containing agents.

2. The method of claim 1, wherein the patient is a breast cancer patient.

3. The method of claim 1, wherein the patient is an ovarian cancer patient.

4. The method of claim 3, wherein the ovarian cancer patient is an epithelial ovarian cancer patient.

5. The method of claim 1, wherein the determining step comprises determining whether the BRCA1 gene obtained from the patient has at least a partial deletion of one or more exons of the BRCA1 gene, provided that the BRCA1 gene with at least a partial deletion of one or more exons maintains the correct reading frame of the BRCA1 gene.

6. The method of claim 5, wherein the partial deletion comprises a partial deletion of exon 11 (a.k.a., exon 10b) of the BRCA1 gene.

7. The method of claim 6, wherein the partial deletion of exon 11 produces a truncated nucleic acid sequence of exon 11 having SEQ ID NO: 27.

8. The method claim 6, wherein the partial deletion of exon 11 comprises BRCA1-Δ11q.

9. The method of claim 8, wherein the BRCA1-Δ11q has the nucleic acid sequence of SEQ ID NO: 26.

10. The method of claim 1, wherein the BRCA1 protein comprises the amino acid sequence of SEQ ID NO: 28.

11. The method of claim 5, wherein the determining step comprises determining whether the BRCA1 gene obtained from the patient has a complete deletion of one or more exons of the BRCA1 gene, provided that the BRCA1 gene with a complete deletion of one or more exons maintains the correct reading frame of the BRCA1 gene.

12. The method of claim 11, wherein the complete deletion comprises a complete deletion of exon 11 (a.k.a., exon 10b) of the BRCA1 gene.

13. The method of claim 12, wherein the complete deletion of exon 11 comprises BRCA1-Δ11.

14. The method of claim 13, wherein the BRCA1-Δ11 has the nucleic acid sequence of SEQ ID NO: 24.

15. The method of claim 1, wherein the BRCA1 protein comprises the amino acid sequence of SEQ ID NO: 25.

16. The method of claim 1, wherein the one or more PARP inhibitors are selected from the group consisting of iniparib, olaparib, niraparib, rucparib, veliparib, BMN 673, CEP 9722, MK 4827, E 7016, and combinations thereof.

17. The method of claim 1, wherein the one or more platinum-containing agents are selected from the group consisting of cisplatin, carboplatin, oxaliplatin, and combinations thereof.

18. The method of claim 1, further comprising isolating the BRCA1 gene from the patient and determining the sequence or structure of the gene.

19. The method of claim 1, wherein the BRCA1 gene comprises DNA, mRNA, or a cDNA obtained from mRNA.


SEQUENCE LISTING (28 sequences)

SEQ ID NO: 1 - DNA, 5592 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 2 - DNA, 54 bp, Homo sapiens
tctggagttgatcaaggaacctgtctccacaaagtgtgaccacatattttgcaa

SEQ ID NO: 3 - DNA, 78 bp, Homo sapiens
attttgcatgctgaaacttctcaaccagaagaaagggccttcacagtgtcctttatgtaagaatgatataaccaaaag

SEQ ID NO: 4 - DNA, 89 bp, Homo sapiens
gagcctacaagaaagtacgagatttagtcaacttgttgaagagctattgaaaatcatttgtgcttttcagcttgacacaggtttggagt

SEQ ID NO: 5 - DNA, 140 bp, Homo sapiens
atgcaaacagctataattttgcaaaaaaggaaaataactctcctgaacatctaaaagatgaagtttctatcatccaaagtatgggctacagaaaccgtgccaaaagacttctacagagtgaacccgaaaatccttccttg

SEQ ID NO: 6 - DNA, 106 bp, Homo sapiens
caggaaaccagtctcagtgtccaactctctaaccttggaactgtgagaactctgaggacaaagcagcggatacaacctcaaaagacgtctgtctacattgaattgg

SEQ ID NO: 7 - DNA, 46 bp, Homo sapiens
gatctgattcttctgaagataccgttaataaggcaacttattgcag

SEQ ID NO: 8 - DNA, 77 bp, Homo sapiens
tgtgggagatcaagaattgttacaaatcacccctcaaggaaccagggatgaaatcagtttggattctgcaaaaaagg

SEQ ID NO: 9 - DNA, 3426 bp, Homo sapiens
ctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttag

SEQ ID NO: 10 - DNA, 89 bp, Homo sapiens
gtgaagcagcatctgggtgtgagagtgaaacaagcgtctctgaagactgctcagggctatcctctcagagtgacattttaaccactcag

SEQ ID NO: 11 - DNA, 172 bp, Homo sapiens
cagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaag

SEQ ID NO: 12 - DNA, 127 bp, Homo sapiens
cagtattaacttcacagaaaagtagtgaataccctataagccagaatccagaaggcctttctgctgacaagtttgaggtgtctgcagatagttctaccagtaaaaataaagaaccaggagtggaaag

SEQ ID NO: 13 - DNA, 191 bp, Homo sapiens
gtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctag

SEQ ID NO: 14 - DNA, 311 bp, Homo sapiens
agggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaattt

SEQ ID NO: 15 - DNA, 88 bp, Homo sapiens
atgctcgtgtacaagtttgccagaaaacaccacatcactttaactaatctaattactgaagagactactcatgttgttatgaaaacag

SEQ ID NO: 16 - DNA, 78 bp, Homo sapiens
atgctgagtttgtgtgtgaacggacactgaaatattttctaggaattgcgggaggaaaatgggtagttagctatttct

SEQ ID NO: 17 - DNA, 41 bp, Homo sapiens
gggtgacccagtctattaaagaaagaaaaatgctgaatgag

SEQ ID NO: 18 - DNA, 84 bp, Homo sapiens
catgattttgaagtcagaggagatgtggtcaatggaagaaaccaccaaggtccaaagcgagcaagagaatcccaggacagaaag

SEQ ID NO: 19 - DNA, 55 bp, Homo sapiens
atcttcagggggctagaaatctgttgctatgggcccttcaccaacatgcccacag

SEQ ID NO: 20 - DNA, 74 bp, Homo sapiens
atcaactggaatggatggtacagctgtgtggtgcttctgtggtgaaggagctttcatcattcacccttggcaca

SEQ ID NO: 21 - DNA, 61 bp, Homo sapiens
ggtgtccacccaattgtggttgtgcagccagatgcctggacagaggacaatggcttccatg

SEQ ID NO: 22 - DNA, 125 bp, Homo sapiens
caattgggcagatgtgtgaggcacctgtggtgacccgagagtgggtgttggacagtgtagcactctaccagtgccaggagctggacacctacctgataccccagatcccccacagccactactga

SEQ ID NO: 23 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 24 - DNA, 2166 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 25 - protein, 721 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 26 - DNA, 2283 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 27 - DNA, 117 bp, Homo sapiens
ctgcttgtgaattttctgagacggatgtaacaaatactgaacatcatcaacccagtaataatgatttgaacaccactgagaagcgtgcagctgagaggcatccagaaaagtatcagg

SEQ ID NO: 28 - protein, 760 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY